(19) United States
(12) Patent Application Publication
(10) Pub. No.: US 2017/0344919 A1
(43) Pub. Date: Nov. 30, 2017

(54) SYSTEM AND METHOD FOR ERGONOMIC MONITORING IN AN INDUSTRIAL ENVIRONMENT

(71) Applicant: Lumo BodyTech, Inc, Mountain View, CA (US)

(72) Inventors: Andrew Robert Chang, Sunnyvale, CA (US); Chung-Che Charles Wang, Palo Alto, CA (US)

(21) Appl. No.: 15/603,906

(22) Filed: May 24, 2017

Related U.S. Application Data
(60) Provisional application No. 62/340,939, filed on May 24, 2016.

(51) Int. Cl. G06Q 10/06; G09B 5/04; A61B 5/00; A61B 5/11; G01P 15/18; G09B 19/00; G09B 5/02

(52) U.S. Cl. CPC G06Q 10/0633; G09B 19/003; G09B 5/04; G09B 5/02; A61B 5/1116; G01P 15/18; A61B 5/486; A61B 5/112; A61B 2562/0219

(57) ABSTRACT

A system and method for ergonomic monitoring in an industrial environment that includes tracking task properties of tasks performed by a worker; for a set of tasks, tracking kinematic activity of a worker during each task instance, which includes collecting kinematic data, processing the kinematic data and generating at least one biomechanical measurement of a task associated kinematic activity; and generating an ergonomic model that associates task properties and kinematic activity.

### CROSS-REFERENCE TO RELATED APPLICATIONS

This Application claims the benefit of U.S. Provisional Application No. 62/340,939, filed on May 24, 2016, which is incorporated in its entirety by this reference.

### TECHNICAL FIELD

This invention relates generally to the field of ergonomic sensing, and more specifically to a new and useful system and method for ergonomic monitoring in an industrial environment.

### BACKGROUND

Recent products have enabled individuals to track and monitor fitness and ergonomics. However, many of these solutions are limited to providing feedback for an individual. While some workers may be able to resolve ergonomic concerns by addressing bad personal habits, other workers are limited by the nature of his or her job. A factory worker that performs strenuous manual tasks through the day not only has little control over the poor ergonomics afforded by an environment, but the factory worker may not be able to specifically identify which aspects of the work are causing problems. Thus, there is a need in the ergonomic sensing field to create a new and useful system and method for ergonomic monitoring in an industrial environment. This invention provides such a new and useful system and method.

### BRIEF DESCRIPTION OF THE FIGURES

FIG. 1 is a schematic representation of a system of a preferred embodiment;

FIG. 2 is a schematic representation of a system used within an environment;

FIGS. 3A-3H are exemplary representations of exemplary single and multi-point sensing configurations;

FIGS. 4A and 4B are exemplary representations of monitoring biomechanical signals during lifting actions with different ergonomics;

FIG. 5 is a flowchart representation of a method of a preferred embodiment;

FIG. 6 is a schematic representation of retrieving static task attributes;

FIG. 7 is a flowchart representation of variations of generating biomechanical measurements;

FIG. 8 is a schematic representation of a generated map of ergonomics;

FIG. 9 is a schematic representation of preemptively generating an alert; and

FIG. 10 is a schematic representation of a workforce logic model.

### DESCRIPTION OF THE EMBODIMENTS

The following description of the embodiments of the invention is not intended to limit the invention to these embodiments but rather to enable a person skilled in the art to make and use this invention.

### 1. Overview

A system and method for ergonomic monitoring in an industrial environment functions to utilize network ergonomic sensing to address ergonomic concerns within an environment and optionally across a set of workers. As workers go about their work, biomechanical attributes can be sensed and correlated with task attributes. For example, the biomechanics of how workers perform a lifting action can be correlated to various locations in a warehouse and/or to the attributes of that task. Similarly, the biomechanics of how workers walk when carrying an item to a loading bay can be analyzed. The system and method can enable a model of the ergonomics of an environment to be used in enhancing ergonomics of a worker and/or altering operations within a facility or in the field.

For workers inside of a warehouse, an industrial plant, a hospital, or other environment, ergonomic risks could be understood from a task-based perspective and used to proactively address them. In one instance, a heat map of ergonomic risk within an environment could be produced. For example, a manager could see that a particular section of a warehouse has an increased risk to poor ergonomics, which may be due to the layout in that area or other factors. In another instance, work distribution can be altered to address ergonomic concerns. For example, work could be redistributed if a particular worker is showing signs of fatigue. In another instance, feedback and guidance could be produced based on the ergonomic aspects of particular tasks. The system and method could be used in providing worker safety training with dynamic monitoring, real-time coaching, and/or automated certification of training completion.

The system and method can employ a variety of ergonomic detection approaches. Preferably, the system and method use at least one kinematic activity monitor device. More specifically, the system and method can additionally include lifting ergonomic detection, posture detection, gait analysis, balance/fall detection, and/or other biomechanical measurements.

### 2. System for Ergonomic Monitoring in an Industrial Environment

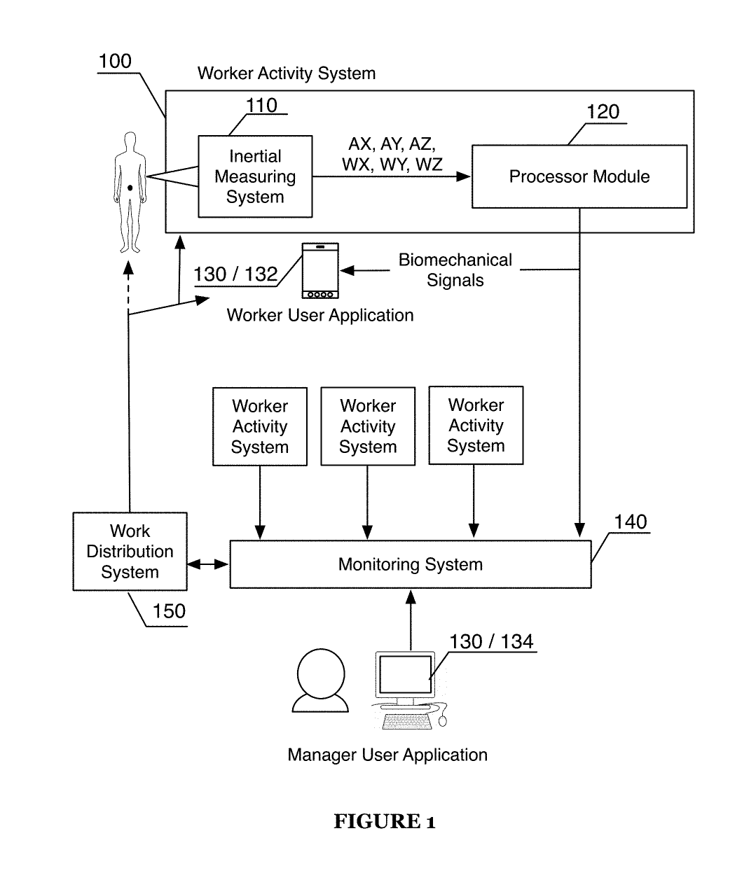

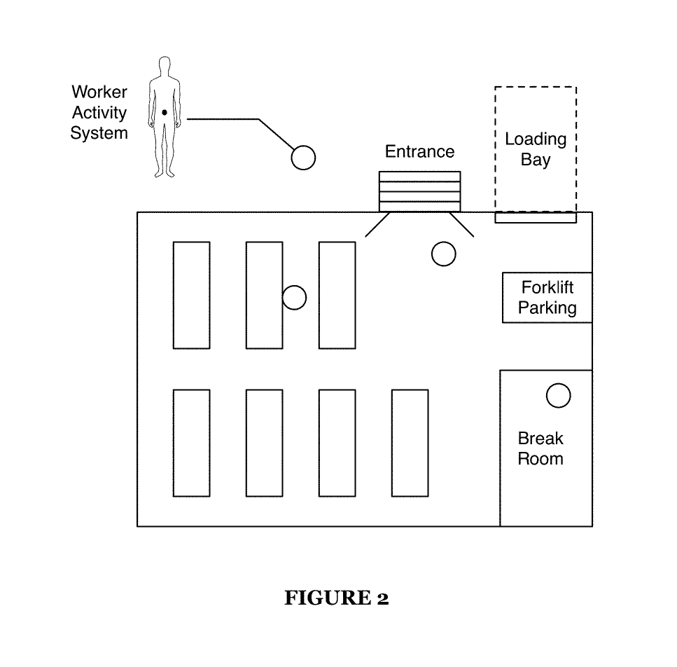

A system for ergonomic monitoring in an industrial environment functions to capture and coordinate ergonomic information collected by at least one worker performing tasks. As shown in FIG. 1, the system can include a set of worker activity systems 100 that include an inertial measurement system 110, a signal processor module 120, and a user application 130, and a monitoring system 140 that can be synchronized with the set of worker activity systems 100. The worker activity systems 100 can be deployed to multiple active workers as shown in FIG. 2. In one variation, the worker activity systems can be distributed to a limited sampling of workers that make up a sub-portion of the workforce. In yet another version, a single worker activity system 100 may be shared by multiple workers. The system is preferably used within some work environment. The work environment may be a substantially closed environment such as a factory or warehouse, but the work environment may alternatively be an open environment such as when monitoring delivery personnel out in the field. The system preferably monitors physical location, the task assigned or performed by the worker, as well as biomechanical attributes of the worker during at least one stage of a task.

The worker activity system 100 can function as an activity monitoring device worn or attached to a worker. The worker activity system 100 can be a standalone device such as a small device that clips on to a belt or clothing. Alternatively, the worker activity system 100 can be integrated into an existing item such as a back support brace, a hardhat, a utility belt, a uniform, and/or any suitable object. The system is preferably used in identifying and acting on identified ergonomic issues.

The worker activity system 100 can be used in monitoring a variety of activities. Preferably, the worker activity system 100 generates one or more biomechanical measurements or signals. A biomechanical signal preferably parameterizes a biomechanical-based property of some action. More particularly, a biomechanical signal quantifies at least one aspect of body position and/or motion during a work related task. If a motion, that motion may be performed once or repeatedly during the task. A task can be any suitable segment of work. In some cases the task is a discrete event or window of time. In other cases, the task can be the general performance of work and can be continuously monitored and inspected as the worker performs a job.

In the industrial application of the system, lifting ergonomics are preferably one particular type of biomechanical signal that can be generated. Various properties of a lifting action can be measured and characterized as a biomechanical signal. These biomechanical signals can be modeled and used in determining if proper lifting technique is used. Lifting biomechanical signals may include angular back displacement during a lift, pelvis vertical displacement, knee angular displacement, difference in vertical displacement of pelvis and neck, and/or other kinematic properties that provide relevant information for the lifting motion. Additional biomechanical signals to help characterize twisting may include angular rotation and angular rotational velocity of the trunk or vertical displacement and counting of climbing up and down a ladder.

The worker activity system 100 may additionally or alternatively provide other biomechanical signals, which can be related to proper biomechanics. Posture, walking gait, balance, fall detection, and/or other biomechanical related measurements can additionally or alternatively be used. For example, in the case of walking, how a participant takes each step can be broken into several biomechanical signals. In a preferred implementation, the system and method preferably operate with a set of biomechanical signals, which may be customized for the particular use case. In the case where the walking gait of a worker is of interest, the biomechanical signals can include step duration, forward velocity properties of the pelvis, vertical oscillation of the pelvis, ground contact time, pelvic rotation, pelvic tilt, pelvic drop, pelvic lateral displacement, stride length, braking, step impact, foot pronation, foot vertical displacement, left foot/right foot stride asymmetry, cadence, cadence variability, and/or any suitable gait attribute.

The pelvis is used as a preferred reference point. The pelvis can have a strong correlation to lower body movements and can be more isolated from upper body movements such as turning of the head and swinging of the arms. An alternative sensing reference point can be used. The sensing point is preferably centrally positioned near the median plane in the trunk portion of the body. Additional sensing points or alternative sensing points may be used depending on the activity. The set of biomechanical signals may form a primitive set of signals from which a wide variety of activities can be monitored and analyzed.

A biomechanical signal can be a function of time. The biomechanical signal can additionally be a real-time signal, wherein each data point is a value that corresponds to some instance of time. Real-time values can be for lifting motions, posture, walking gait strides, and/or any suitable type of biomechanical signals. The real-time value can be a current value for the biomechanical property or a running average. For example real-time stride time can be averaged over a window spanning a defined set of steps (e.g., 4 steps). Averaging, smoothing, and other error correcting processes may be applied to a real-time signal.

In one preferred implementation, the worker activity system 100 includes at least one inertial measurement system 110, which may additionally include a housing, the signal processor module 120, memory/storage, and/or a communication component. The worker activity device 100 can additionally include any suitable components to support computational operation such as a processor, RAM, an EEPROM, user input elements (e.g., buttons, switches, capacitive sensors, touch screens, and the like), user output elements (e.g., status indicator lights, graphical display, speaker, audio jack, vibrational motor, and the like), communication components (e.g., Bluetooth LE, Zigbee, NFC, Wi-Fi, cellular data, and the like), and/or other suitable components. The worker activity system 100 in one variation can relay data to a worker user application 130 on a personal computing device. In an alternative embodiment, the system may include a worker activity device 100 without a user application 130. The worker activity device 100 may directly communicate with the monitoring system. For example, kinematic data or biomechanical signal data could be sent over Wi-Fi or a cellular network. In another variation, the worker activity system 100 could be implemented on a personal computing device such as a mobile phone and/or a wearable computer.

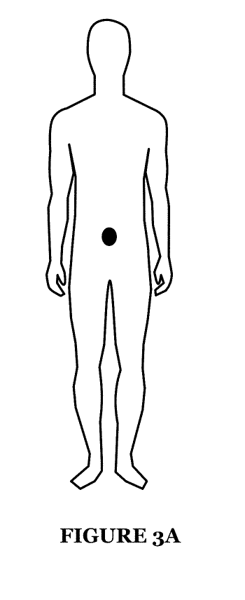

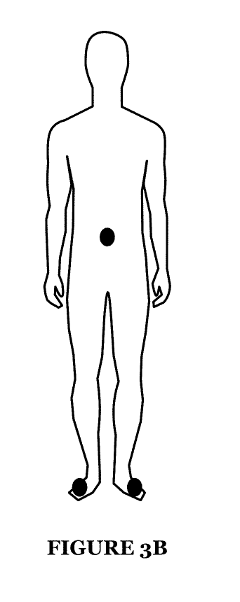

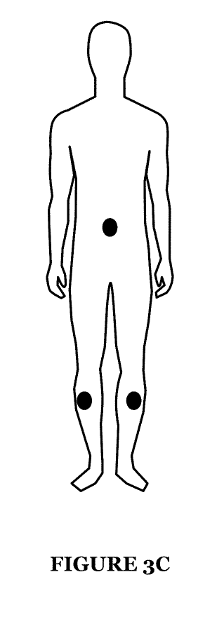

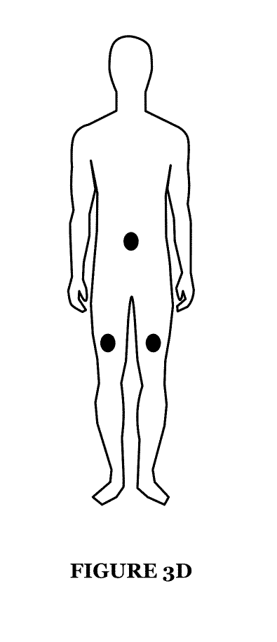

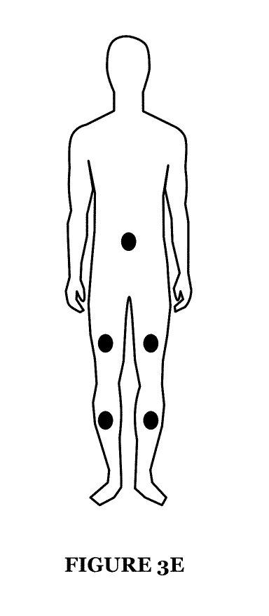

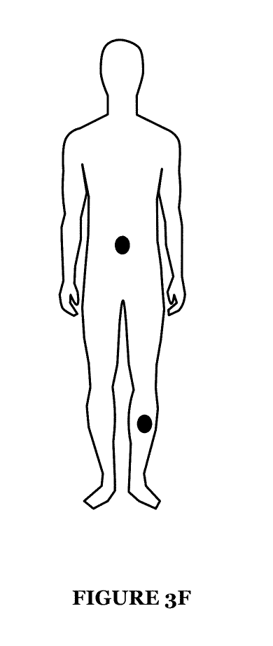

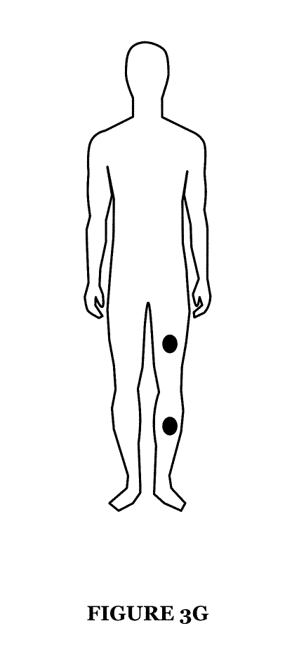

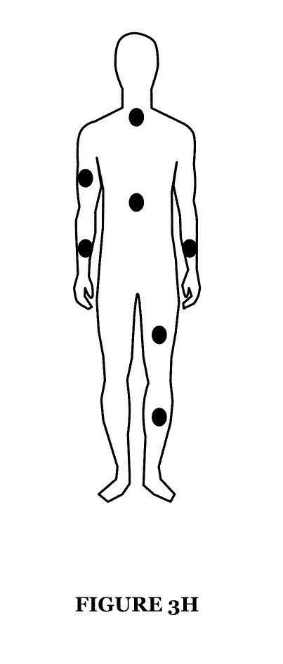

In one variation, the system includes a single worker activity system 100. The worker activity system 100 is preferably positioned in the waist region, and more specifically, the worker activity system 100 can be positioned along the back in the lumbar or sacral region as shown in FIG. 3A. A single worker activity system 100 can be used in lifting and bending analysis, left and right foot detection, stride analysis, braking analysis. The single point of sensing from the worker activity system 100 can be positioned at any suitable alternative position.

In another variation, the system uses a multi-point sensing approach, wherein a set of inertial measurement systems 110 of measure motion at multiple points. The inertial measurement systems no can be integrated into distinct activity monitoring devices wherein the worker activity system 100 includes multiple communicatively coupled activity monitoring devices. The points of measurement may be in the waist region, the upper leg, the lower leg, the foot, and/or any suitable location. Other points of measurement can include the upper body, the head, or portions of the arms. Various configurations of multi-point sensing can be used for sensing biomechanical signals. Different configurations may offer increased resolution, more robust sensing of one or more signals, and for detection of additional or alternative biomechanical signals. A foot activity monitor variation of a worker activity system 100 could be attached to or embedded in a shoe. A shank or thigh activity monitor could be strapped to the leg, embedded in an article of clothing, or positioned with any suitable approach. In one preferred variation of multi-point sensing, the system includes a pelvic activity monitor device and a right and left lower leg activity monitor device. The right and left lower leg worker activity system 100 may be coupled to the foot as shown in FIG. 3B or the shank of each of the legs as shown in FIG. 3C. The lower leg worker activity system 100 can provide additional kinematic insights into how the extremities of the leg are moving. In another variation, the system can include activity monitor devices of the worker activity system 100 on the right and left thigh as shown in FIG. 3D. The thigh activity monitor devices can be used in combination (as shown in FIG. 3E) or as an alternative to the lower leg activity monitor devices. The thigh activity monitor devices can be used to give relative rotation and motion between the thigh and the foot area and/or between the thigh and the pelvic region. While multi-point sensing preferably uses symmetrical sensing for both legs, but one alternative may use a single additional sensing point on the leg as shown in FIG. 3F. In the multiple participants embodiment, multiple activity monitor configurations may be supported. A second individual may wear a first and second activity monitor about the knee to measure knee mobility biomechanical signals as shown in FIG. 3G. Other sensing points may additionally or alternatively be used such as the upper core, neck, hands, and/or other points as shown in FIG. 3H.

Multiple points of sensing can be used to obtain motion data that provides unique motion information that may be less prevalent or undetectable from just a single sensing point. Multiple points can be used in distinguishing alternative biomechanical aspects and/or to detect particular biomechanical attributes with more resolution or consistency. Multiple points may be used for detecting foot gait attributes, knee flex angle, and/or distinguishing between right and left leg actions. Single point sensing may additionally be applied to right and left leg attributes, upper core body or arms. The multiple points can be used to obtain clearer signals for particular actions such as when a user bends to pick up a heavy object or rotates his body left or right. Multiple points can additionally be used in providing relative kinematics between different points of the body. The relative angular orientation and displacement can be detected between the foot, thigh, pelvic, thoracic and neck region. Similarly, relative velocities between a set of activity monitor systems can be used to generate particular biomechanical signals.

The inertial measurement system 110 functions to measure multiple kinematic properties of an activity. The inertial measurement system 110 preferably includes at least one inertial measurement unit (IMU). An inertial measurement unit can include at least one accelerometer, gyroscope, magnetometer, or other suitable inertial sensor. The inertial measurement unit preferably includes a set of sensors aligned for detection of kinematic properties along three perpendicular axes. In one variation, the inertial measurement unit is a 9-axis motion-tracking device that includes a 3-axis gyroscope, a 3-axis accelerometer, and a 3-axis magnetometer. The sensor device can additionally include an integrated processor that provides sensor fusion in hardware, which effectively provides a separation of forces caused by gravity from forces caused by speed changes on the sensor. The on-device sensor fusion may provide other suitable sensor conveniences. Alternatively, multiple distinct sensors can be combined to provide a set of kinematic measurements.

A worker activity system can additionally include other sensors such as an altimeter, GPS, or any suitable sensor.

Additionally, the system can include a communication channel to one or more computing devices with one or more sensors. For example, an inertial measurement system no can include a Bluetooth communication channel to a smart phone, and the smart phone can track and retrieve data on geolocation, distance covered, elevation changes, and other data.

Preferably, the system can include a positioning system, which may use GPS for global positioning, Bluetooth beacons, RF-based triangulation for environmental positioning, and/or any suitable system that can generate location information within a global or local environment. In one variation, the positioning system can be integrated with the worker activity device 100. In another variation, the positioning system can use a location service of a connected device such as the GPS or Bluetooth service of a mobile phone.

The signal processor module 120 functions to transform sensor data generated by the inertial measurement system no. The signal processor module 120 preferably can include processing modules that output one or more biomechanical signals. Preferably, the biomechanical signals include a biomechanical signal related to lifting ergonomics. The biomechanical signals may additionally or alternatively relate to posture and/or moving (e.g., walking or carrying). In a variation using multiple sensing points, the signal processor module 120 for one of the activity monitor devices can be customized for the particular sensing point. A signal processor module 120 of a foot activity monitor device may be customized for walking biomechanical signals, while a signal processor for the thigh and shin may be customized for lifting related biomechanical signals. More preferably, the signal processor module 120 of activity monitor devices at different locations can be substantially similar, but can be set to operate in different modes. For example, different modes of kinematic activity tracking and/or ergonomic models can be enabled in the signal processor module 120 of an activity monitor device depending on its positioning on the user. The kinematic activity tracking and/or ergonomic models could additionally be processed partially or fully on a user application 130 or in a remote server.

The signal processor module 120 can be integrated with the inertial measurement system 110. For example, a wearable device with a battery, a communication module, and some form of user control can generate the biomechanical signals on a single device. The signal processor module 120 may alternatively be application logic operable on a secondary device such as a smart phone. In this variation, the signal processor module 120 can be integrated with the user application 130. In yet another variation, the signal processor module 120 can be remote processor accessible over the network. For example, biomechanical signals may be generated in the cloud-based monitoring system, which can provide remote processing. Remote processing can enable large datasets to be more readily leveraged when analyzing kinematic data.

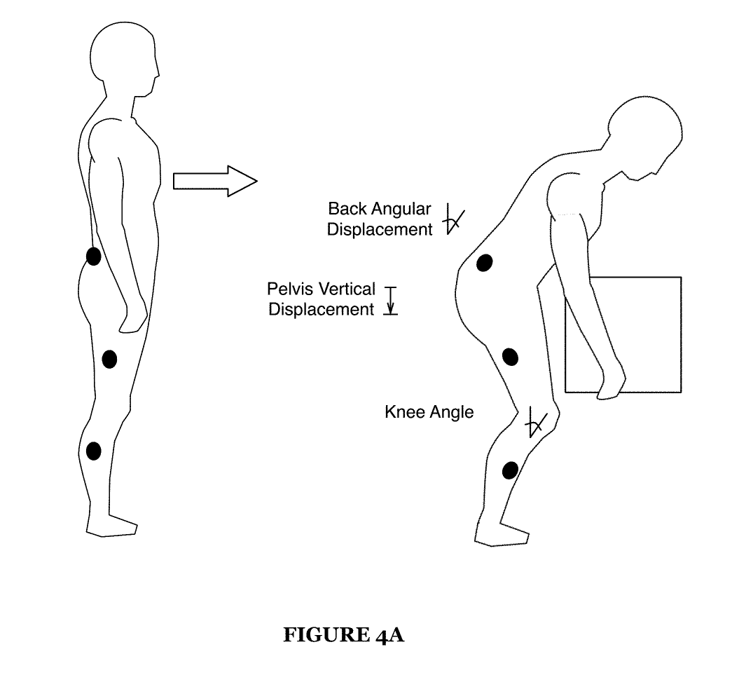

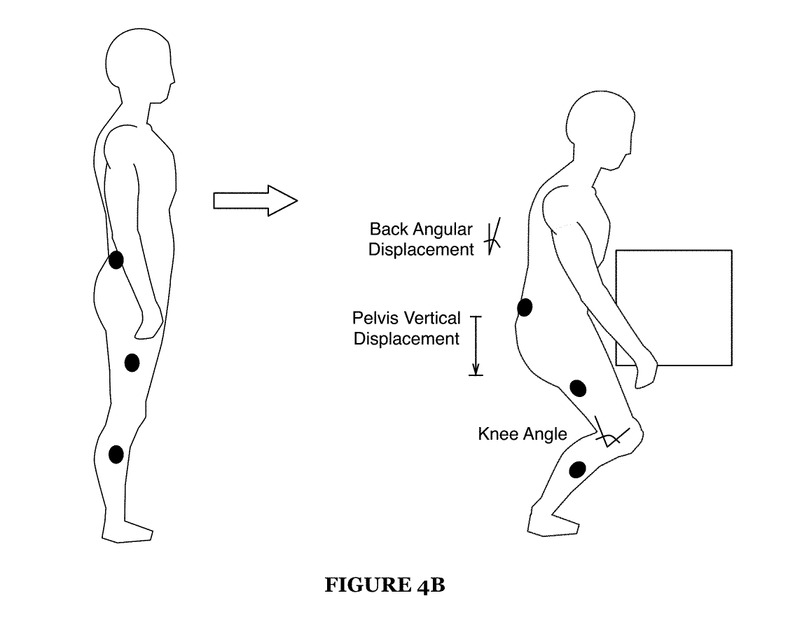

In a variation with multiple sensing points, a set of signal processor modules 120 can be included in a plurality of activity monitor devices worn by a worker. The generation of the biomechanical signals can be completed on each of the activity monitor devices. Alternatively, kinematic data, processed data, and/or a partial set of biomechanical signal data can be communicated between the set of activity monitor devices such that data from multiple sensing points can be used in generating a single biomechanical signal. For example, an upper thigh activity monitor device and a shin activity monitor device can cooperatively determine knee bending rotation by measuring respective angular displacement and then generating a resulting knee rotation as shown in FIGS. 4A and 4B. Similarly, an activity monitor device positioned on the pelvis or along the back can be used with one or more activity monitor devices on the thigh or below the hip to generate a hip rotation. In yet another variation, displacements (e.g., vertical displacement, forward displacement, etc.) can be accounted for by multiple activity monitor devices. Modeled or predicted biomechanical motion can be generated using relative displacements. For example, if an activity monitor on the upper torso has a fourteen-inch vertical displacement and the pelvis has a one-inch displacement, the modeled biomechanical motion can indicate a lifting motion using the back. Whereas if the upper torso has a fourteen inch vertical displacement and the pelvis similarly has a fourteen inch. As an exemplary scenario of monitoring lifting ergonomics, bad lifting ergonomics may be characterized as a larger angular displacement of the pelvis, a smaller vertical displacement of the pelvis, and/or a small angular displacement of the knee as shown in FIG. 4A, and good lifting ergonomics may be characterized by smaller angular displacement of the pelvis, a larger vertical displacement of the pelvis, and/or a larger angular displacement of the knee as shown in FIG. 4B.

One variation can include a right foot and left foot activity monitor device communicating with a parent pelvic activity monitor device. The left and right activity monitor devices can generate specific biomechanical signals. A subset of the biomechanical signals may be used by the parent activity monitor to generate an additional or alternative biomechanical signal. The parent pelvic activity monitor device can communicate data to a user application 130 on a user device. Alternatively, each of the activity monitor devices may directly communicate with the user application 130. The user application 130 can facilitate completion or generation of all or some biomechanical signals and ergonomic models.

The user application 130 functions to provide a user interface for the worker and/or the manager. The system can include a worker user application 132, which is preferably used primarily by a monitored worker. The system can additionally or alternatively include a manager user application 134, which is preferably used by one or more administrators overseeing activity of a set of workers. The user application 130 can be any suitable type of user interface component. Preferably, the user application 130 is a graphical user interface operable on a user computing device. The user computing device can be a smart phone, a desktop computer, a TV based computing device, a wearable computing device (e.g., a watch, glasses, etc.), or any suitable computing device. The user applications 130 can communicate with one or more worker activity systems.

A worker user application 132 can manage operation of a single worker activity system 100. For example, the biomechanical signal information for a connected worker activity system 100 can be represented in a user interface so that a worker can review personal information. The worker user application 132 may show a lifting score, carrying score, posture score, and/or other qualitative reports relating biomechanics and/or ergonomics. The worker user application 132 can additionally provide detailed metrics such as back angle rotation during a lift and percentage of foot bias when carrying an object. The worker user application 132 can additionally display alerts, provide active feedback (e.g., vibrate or audio cues), or other forms of feedback to notify or warn a worker. For example, an audio or haptic warning cue may be played if a worker is improperly lifting an object, twisting in a non-ergonomic manner, and/or climbing down a ladder too quickly. Such feedback can be predictive, real-time, or as a post-performance review. The predictive feedback can be used to alert a worker to potential performance risk, which may be based on personal performance or group performance. For example, if a set of workers has recently been using bad ergonomics when lifting items of a particular weight, a warning message may be played before lifting such an object. The worker user application 132 may additionally or alternatively be used for other purposes. In one example, the worker user application 132 may enable specific ergonomic models that measure the biomechanical quality of the task or associated risk before providing worker feedback. In another example, the worker user application 132 may primarily be used by the worker for inspecting and completing work tasks such as seeing location of an item for shipping, scanning the item, and printing a shipping label.

A manager user application 134 preferably has access to activity information relating to the set of workers. The manager user application 134 can show group analytics and visualizations. The manager user application 134 may additionally allow inspection of a selected worker's performance and injury risk profile, as well as physical areas within the warehouse with most safety risk. The manager user application 134 may be used for setting and updating system configuration. For example, a manager may change the threshold for issuing ergonomic feedback.

The monitoring system 140 of a preferred embodiment, functions as a centralized manager of ergonomic monitoring. The monitoring system 140 preferably collects data from a set of workers. In one variation, the monitoring system 140 is a cloud-computing platform, with which various manager user applications communicate. The worker activity systems 100 can collect the data and transfer the data to the monitoring system. In another variation, the monitoring system 140 can be a local monitoring system, which may be the same application as the manager user application 134. For example, an application running on a desktop computer could be used as the centralized manager of data for the set of workers. Processing and analysis of the data can be performed at the monitoring system. The monitoring system 140 can additionally include an application programming interface such that other systems and services may access information and/or trigger changes to the system.

In one variation, the system of a preferred embodiment can include a work distribution system 150. The work distribution can include or be integrated with a computerized subsystem used in directing and/or assigning work and tasks across a workforce. For example, in a shipping warehouse, this may include the system used in directing workers what products to select for shipping. The work distribution system 150 preferably interfaces with the monitoring system 140, wherein activity and ergonomic impact of work tasks can be considered when generating work assignments. In one variation, the work distribution system 150 can select workers according factors relating to ergonomics and physical fatigue. In another variation, the work distribution system 150 can automatically update the nature of the work assignment. In one instance, if the monitoring system 140 identifies an area of the warehouse has a high ergonomic risk. Then the items normally positioned in that area can be redistributed to regions that may be safer from an ergonomic perspective. For example, several large, heavy items may be placed on a high shelf, but the work distribution system 150 could automatically identify a new storage position for those items at a lower and more accessible region. The work distribution system 150 could transition the item organization of the warehouse through a set of different work orders. In one variation, the work distribution system 150, communicates tasks and work assignments through the worker user applications. In another variation, the work distribution system 150 interfaces with alternative systems or services for communicating task directives.

### 3. Method for Ergonomic Monitoring in an Industrial Environment

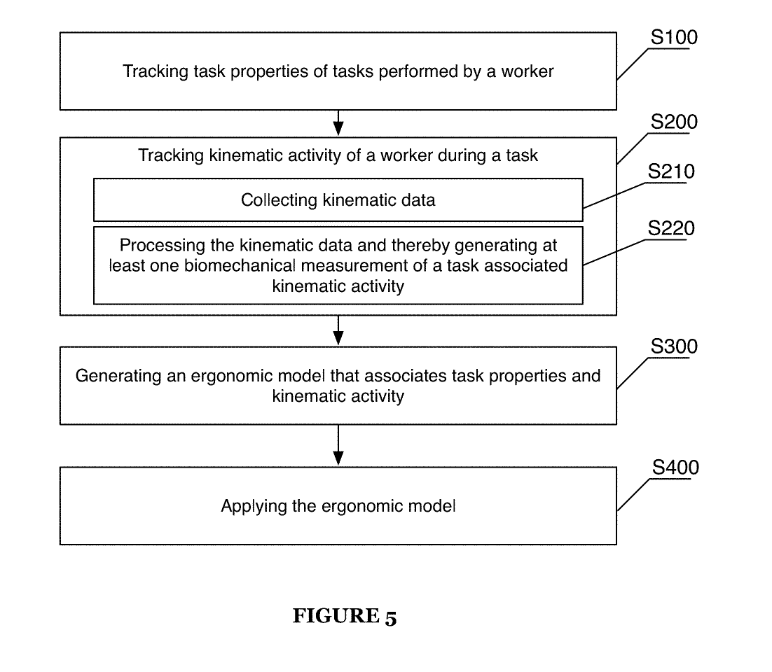

As shown in FIG. 5, a method for tracking environmental ergonomics of a preferred embodiment can include tracking task properties of tasks performed by a worker S100, tracking kinematic activity of a worker during a task S200, generating an ergonomic model that associates task properties and kinematic activity S300; and optionally applying the ergonomic model S400.

The method functions to detect how workers in a workforce move within a particular setting and then apply that to transform the ergonomics of workers and/or to address ergonomic impact of a work environment. More preferably the method can transform the ergonomic impact in an industrial environment or other form of environment of a worker.

The method can be used in generating active feedback for workers, for elevating ergonomic risks and insights before an incident occurs (e.g., a worker injury), providing training on proper work ergonomics, characterizing ergonomic and work patterns, dynamically altering the operations within an environment taking into account the ergonomic risk and impact of the work, and/or applied in any suitable manner.

In one variation, the method (and more specifically blocks S100 and S200) can be implemented in connection to a single worker. The collected data and application of the resulting ergonomic model can function for a single worker.

In another variation, the method (and more specifically blocks S100 and S200) can be implemented across a plurality of individuals interacting within the environment. For example, all the workers in a warehouse can be equipped with ergonomic detection equipment. In some implementation variations, the workforce may comprise of a single worker. This variation of the method may be used by a company to improve and alter the ergonomic performance of a workforce (e.g., reducing ergonomic related risk for workers). A method used across a work force can include tracking task properties of tasks performed by workers of the workforce; for a set of tasks performed by workers of the workforce, tracking biomechanical measurements of the set of workers during tracked task instances; processing the biomechanical measurements into an ergonomic measurements; and generating an ergonomic model that associates task properties and ergonomic measurements across a workforce.

In one variation, the method can be applied within a limited, confined environment such as a factory, a warehouse, a shipping yard, a construction site, a hospital, and/or any suitable confined environment. The method may alternatively be applied to an open environment. For example, the method could be used in monitoring the ergonomic risks for delivery personnel operating out of a shipping vehicle while visiting various sites along a shipping route.

In another variation, the tasks of a worker are not explicitly tracked, and the tracking of the kinematic activity of a worker can happen continuously independent of any association with task properties. Various kinematic activities such as lifting actions, ladder climbing, posture, gait analysis, and/or other modes of analyzing kinematics of a worker can be executed and used to generate feedback and/or analytics. In some variations, these different modes of kinematic activity tracking and/or ergonomic model analysis can be dynamically enabled by classifying kinematic data signals, using time of day, and/or other properties to switch operating modes.

As a list of exemplary use cases, the method can be applied within a warehouse, shipping and courier industries, a manufacturing plant, construction site, military operations, law enforcement operations, hospital operations (e.g., assessing ergonomics of doctors, nurses, orderlies, technicians, etc.), insurance and worker compensation analysis, and/or any suitable scenario that may involve one or more workers performing physical tasks.

The method preferably employs biomechanical tracking techniques based on inertial sensing as described herein to provide qualitative ergonomic insights. Herein, biomechanical sensing is used as the primary biometric property, but additional biometric signals could be collected, processed and used in combination with the biomechanical. For example, biometric sensing such as heart rate, respiratory rate, blood chemistry and/or other biometric properties can additionally be collected and used. Alternatively, the method may be used with a wide variety of kinematic sensing and ergonomic detection approaches or systems. Preferably, a system substantially similar to the system described above is used, but any suitable system may alternatively be utilized.

Block S100, which includes tracking task properties of tasks performed by a worker, functions to collect contextual information during a task that can be correlated with a kinematic activity performed during that task. Task properties preferably provide contextual information around a task. In one particular implementation, location of a particular task event can be used to model the ergonomics of the work performed. Similarly, the path of a task that involves locomotion (e.g., carrying or carting an object to some destination) is another form of base task properties that can be used in understanding the ergonomics of those tasks. In one variation, the set of task properties may be customized according to the particular use-case or environment of the particular implementation. The task properties are preferably synchronized within operation of the method such that biomechanical measurements can be associated with particular tasks and/or task properties.

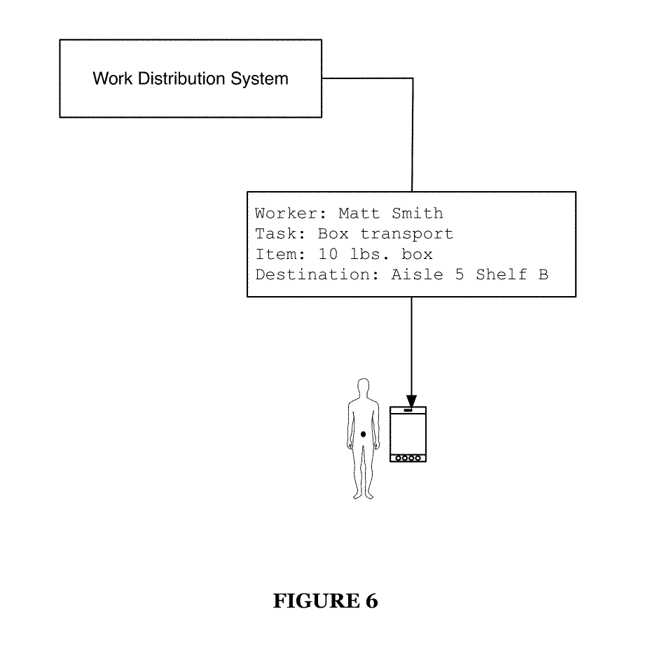

Monitoring properties of a task can include retrieving static task attributes. More specifically, monitoring properties of a task includes retrieving current task assignments and task attributes associated with each task assignment as shown in FIG. 6. The task assignments are preferably retrieved from a work distribution system. Task attributes can be characteristics that can be inherited in performing the task and may be known beforehand based on the objective of the task. The task attributes may be provided from a work management system. For example, a picker within a warehouse may be assigned to fetch an item and prepare the item for shipping. The ergonomics and biomechanical actions during that task can be monitored and correlated with properties of that task. A work management system may include information relating to the item such as the dimensions and the size, the location within the warehouse where the item is to be picked up, and the location where the item is to be moved.

Task attributes may include task type such as “lifting”, “moving”, “opening”, “climbing ladder”, “climbing stairs”, “walking on ramp”, “carrying object” or any suitable type of task. Task attributes may include subject attributes such as object dimensions, object weight, and/or other attributes of objects acted upon by the worker during a task. Task attributes may include position properties, which may be a facility location (e.g., aisle 6 section A), relative position (e.g., x: 312.5 ft., y: 82 ft.), global position (e.g., GPS determined coordinates), object position (e.g., top row of rack), or any suitable property relating to position. Task attributes can additionally or alternatively include any suitable pre-defined attributes of a task, which may be specialized for the particular use case.

Task attributes may additionally include worker state, which can characterize properties of the worker at the time of the task. Worker state can include duration of employment, time worked prior that day leading up to the task, number of tasks performed prior to that task, type of tasks performed up to that task, demographic information, employment history (e.g., training, job level, etc.), and/or any suitable information. By collecting this information and associating it with kinematic activity, the method can be used in applications where, daily workload can be managed according to ergonomic/health risk. Properties of a task can additionally be retroactively mapped to a timestamp and associated with kinematic data. For example, task properties may be entered by a manager, a worker, or synchronized from external data system after the task is completed.

Monitoring properties of a task can additionally include sensing task related data during the task. Additional sensing capabilities can be used to detect aspects of the worker, an object, or the environment. For example, sensing task related data can include tracking position of the worker. Tracking position may use GPS, Wi-Fi positioning system, Bluetooth beacons, RF-based positioning system, and/or any suitable mechanism to track position of the worker as they work. Such information may be collected from a sensing device, by an application operable on a computing device. In one implementation, lifts and bending actions are tracked. The time of the action, the location of the action, and a kinematic analysis of the action (e.g., the lift or bending) can be logged.

Accordingly, a method variation with tracked position can include tracking location of the worker through a positioning system, which functions to track location of a worker. Preferably, the location of the worker can be collected alongside other task properties such as a timelog of tasks and their attributes. An alternative implementation of the method may simply track location and kinematic activities of the worker. Tracking location of the worker can include tracking a location of the worker within a defined environment through a local positioning system such as using a RF-based or Wi-Fi positioning system. Tracking location of the worker may additionally or alternatively include tracking a location of the worker within a global positioning system. A GPS module or a location service of a mobile device may be used in collecting global positioning.

As discussed below, the task properties (e.g., location information and/or task attributes) can be used in dynamically altering the type of kinematic activity processing, in generating the ergonomic model, and/or applying the ergonomic model.

Block S200, which includes tracking kinematic activity of a worker during a task, functions to characterize movement properties of a worker associated with a task. Tracking kinematic activity preferably includes: at an inertial measurement unit attached to the worker, collecting kinematic data S210, and processing the kinematic data and thereby generating at least one biomechanical measurement of a task associated kinematic activity S220.

The biomechanical measurements are preferably ones that relate to one or more kinematic activities relevant to a task such as posture, lifting actions, walking gait, balance/fall detection, and/or other suitable kinematic activities. Depending on the particular application of the method, different kinematic activities may be of interest.

A kinematic activity is preferably monitored using an inertial measurement system as described above. Some biomechanical measurements of a kinematic activity may be tracked and detected by monitoring at least one metric of an inertial measurement system over time. Other biomechanical measurements of a kinematic activity may be generated through processing of two or more metrics of a single inertial measurement system. In other variations, a biomechanical measurement of a kinematic activity may be generated through processing metrics from two or more activity tracking systems on a worker (e.g., one on the pelvis and one on the knee).

In one variation, the kinematic activity can be continuously tracked over a particular work interval (e.g., an hour, one work day, a week, a month, a year, etc.). In another variation, tracked kinematic activity may be segmented by discrete tasks such as walking a particular package from a shipping vehicle to the front door of a delivery destination. Accordingly, the method may include triggering tracking of kinematic activity. Kinematic activity tracking can be triggered based on digitally signaled work activity. In the shipping example above, a door-delivery activity may be triggered when a delivery person stops at a particular destination as detected by a GPS-enabled application or, alternatively, when a package is scanned for leaving a truck.

Block S210, which includes collecting kinematic data, functions to sense, detect, or otherwise obtain sensor data relating to motion of a worker. The kinematic data can be collected with an inertial measurement system that may include an accelerometer system and/or a gyroscope system. Preferably, the inertial measurement system includes a three-axis accelerometer and gyroscope. The kinematic data is preferably a stream of kinematic data collected over periods of time when a task is performed. The kinematic data may be collected continuously but may alternatively be selectively activated prior to a task.

In one variation, data of the kinematic data is raw, unprocessed sensor data as detected from a sensor device. Raw sensor data can be collected directly from the sensing device, but the raw sensor data may alternatively be collected from an intermediary data source. In another variation, the data can be pre-processed. For example, data can be filtered, error corrected, or otherwise transformed. In one variation, in-hardware sensor fusion is performed by an on-device processor of the inertial measurement unit. The kinematic data is preferably calibrated to some reference orientation. In one variation, automatic calibration may be used as described in U.S. patent application Ser. No. 15/454,514 filed on Mar. 9, 2017, which is hereby incorporated in its entirety by this reference.

Any suitable pre-processing may additionally be applied to the data during the method. In one variation, collecting kinematic data can include calibrating orientation and normalizing the kinematic data.

An individual kinematic data stream preferably corresponds to distinct kinematic measurements along a defined axis. The kinematic measurements are preferably along a set of orthonormal axes (e.g., an x, y, z coordinate plane). As described below, the axis of measurements may not be physically restrained to be aligned with a preferred or assumed coordinate system of the activity. Accordingly, the axis of measurement by one or more sensor(s) may be calibrated for analysis by calibrating the orientation of the kinematic data stream. One, two, or all three axes may share some or all features of the calibration, or be calibrated independently. The kinematic measurements can include acceleration, velocity, displacement, force, rotational acceleration, rotational displacement, tilt/angle, and/or any suitable metric corresponding to a kinematic property of an activity. Preferably, a sensing device provides acceleration as detected by an accelerometer and angular velocity as detected by a gyroscope along three orthonormal axes. The set of kinematic data streams preferably includes acceleration in any orthonormal set of axes in three-dimensional space, herein denoted as x, y, z axes, and angular velocity about the x, y, and z axes. Additionally, the sensing device may detect magnetic field through a three-axis magnetometer.

Calibrating the kinematic data can involve standardizing the kinematic data and calibrating the kinematic data to a reference orientation such as a coordinate system of the participant. The nature of calibration can be customized depending on the task and/or kinematic activity. For example, in a walking task, normalizing the set of kinematic data streams can include adapting orientation of kinematic data sensing to a participant orientation, determining a base pelvic tilt position, and segmenting at least a subset of the kinematic data streams by detected steps. The inertial measurement unit is preferably part of a handheld device that can be attached or otherwise fixed into a certain position during an activity. That position can be static during the activity but may also be perturbed and change. Preferably, the inertial measurement unit is positioned in the waist region and more specifically in the lumbar or sacral region of the back. Additional inertial measurement units can be positioned at varying points to provide kinematic data streams for other portions of the body. The foot, the shank of the leg, and the thigh are three optional points. Inertial measurement units can capture kinematic data streams for each of a right leg and a left leg.

Block S220, which includes processing the kinematic data and thereby generating at least one biomechanical measurement of a task associated kinematic activity, functions to produce a metric used in assessing worker ergonomics and biomechanics during a task. A biomechanical measurement is a metric or signal that preferably characterizes at least one aspect of how a user is moving or positioned. One or more different biomechanical measurements may be produced depending on the use case and/or the current operating state.

The biomechanical measurements may characterize the motion patterns of a particular body part. For example, the motion of a foot, a knee, pelvis, trunk or any suitable body part can be monitored and characterized in one or more biomechanical measurements. The biomechanical measurements may alternatively characterize the biomechanical relationship of multiple body parts. For example, the biomechanical measurements can show imbalances in a user's gait or a comparison of mobility between two joints. The biomechanical measurements may alternatively characterize the mobility quality of the particular body part, joint, or overall walking gait over time.

In a preferred implementation a set of biomechanical measurements are generated, which functions to parameterize a set of primitives from which the motion properties of a task can be monitored and acted on. The biomechanical measurements are preferably specific for particular activities and/or use cases. As will be described below, the method may enable dynamic selection of different operating modes so that an activity monitoring device can adapt to tasks of a worker. Alternatively, the set of biomechanical measurements may be preconfigured and not dynamically selected.

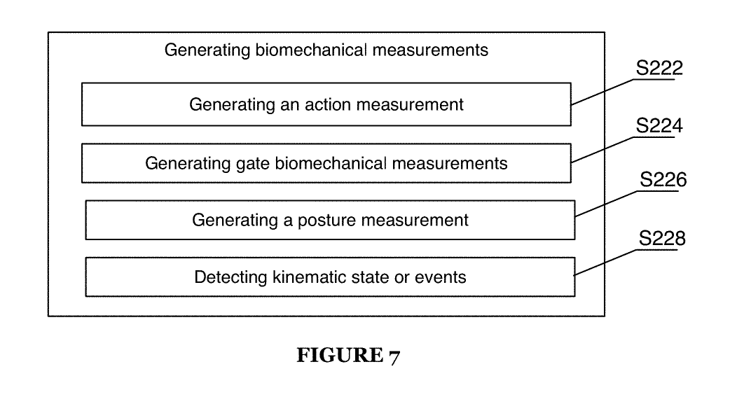

In some variations, generating the biomechanical measurements can include generating an action measurement 5222, generating gait biomechanical measurements S224, generating a posture measurement S226, and/or detecting kinematic state or events S228 as shown in FIG. 7. Action measurements can include various actions like lifting actions, bending actions, twisting actions, ladder climbing actions, stair climbing actions, crawling actions, lying down actions, jumping actions, and the like. These different biomechanical measurements could be generated continuously for every task or may alternatively be selectively activated when appropriate.

Alternatively, the method can include selecting at least one of a set of kinematic activities (e.g., lifting, walking, standing, carrying etc.) and generating biomechanical measurements associated with that kinematic activity. In one variation the selection of the kinematic activity can be based in part on the task properties. A work distribution system may indicate the type of task currently assigned to a worker, and then the method will generate biomechanical measurements associated with the kinematic activities associated with the currently assigned task. For example, a work distribution system may indicate a worker is transporting a box of items to storage and so lifting related biomechanical measurements related as posture, lifting motion, and/or the worker's gait can be collected during that task.

In a related variation, the selection of the kinematic activity can be based in part on a current location of the worker. The location can be indicated by a position system. This can be used so that the kinematic activities of a worker can be changed based on location. Associations of locations and kinematic activities may be preconfigured. In one implementation, a closed environment may have a map created where different kinematic activities can be plotted and assigned to different subregions of the environment. Associations of locations and kinematic activities may alternatively be dynamically determined based on an existing ergonomic model. Regions with higher ergonomic risk (e.g., where previously measured kinematic activity was indicative of bad or concerning ergonomics) can trigger tracking of kinematic activity.

Selection of a kinematic activity could additionally or alternatively be selected based on active monitoring of kinematic data. For example, different actions such as lifting, bending, twisting, climbing ladders, and the like can be detected and used in selecting the detected action or actions.

Block S222, which includes generating an action measurement, functions to measure biomechanics as a worker performs some action. As discussed, the action measurement can include one or more types of generated measurements such as generating a lifting measurement, a bending measurement, a twisting measurement, a ladder climbing measurement, a stair climbing measurement, a crawling measurement, lying down measurement, a jumping measurement, and/or measurements of other suitable actions. Generating an action measurement can be used to detect the occurrence of the action but can additionally provide one or more metrics on how that action was performed.

Generating a lifting measurement, for example, functions to measure biomechanics as a worker lifts some object. Generating a lifting measurement may be achieved in a variety of approaches. In one variation, generating a lifting measurement can include tracking comparative usage of the lower body (e.g., legs) and the upper body (e.g., back) during a lifting action. Proper lifting ergonomics is generally characterized by lifting with one's legs as opposed to one's back. During a lifting action, the user preferably bends one's knees to provide the vertical displacement when getting in position to grab an object and when lifting the object. The upper body is preferably not used to provide the lifting force. The upper body during proper lifting ergonomics generally does not undergo significant rotation (e.g., does not bend at the waist) and the vertical displacement is preferably substantially consistent across points on the upper body (e.g., the pelvis experiences a similar vertical displacement as the neck). When tracking comparative usage of the upper body and lower body, kinematic data is preferably collected and monitored for the lower body and the upper body. The kinematic data can provide biomechanical measurements relating to rotation and/or displacement at various points. Detecting rotation of the knees or hip and/or the vertical displacement of the pelvis may be used for the lower body. Detecting rotation of the back and/or vertical displacement of one or more points along the back may be used for the upper body. The kinematic data may be compared or the kinematic data may provide one type of biomechanical measurement used in characterizing lifting ergonomics.

Generating a bending measurement can include detecting some biomechanical property related to how a worker bends. Bending can be a measure of the angle of change of the pelvis in the sagittal plane and may also include vertical and forward/backward displacement of the pelvis as a worker may not just bend from the hips but bends from the knees. Generating a bending measurement can include generating various bending related parameters, which may include the change in angle, angular velocity change, displacement of pelvis in the forward/backward and vertical planes, the time to complete a bend, counting the number of bends, and/or other suitable parameters.

In one implementation generating a bending measurement can include detecting and segmenting a bending event and calculating the angle difference of the pelvis relative to the gravity vector in the forward/backward plane at the beginning of the bend and at the maximum bend angle. Another parameter is to measure the displacement of the pelvis in the vertical and forward backwards plane. Segmentation and error correction as discussed in U.S. patent application Ser. No. 15/420,105 filed on Jan. 31, 2017 and U.S. patent application Ser. No. 15/282,998 filed on Sep. 30, 2016, which is hereby incorporated in its entirety, can be used to analyze each individual bend. The segmentation can occur during a time period between two low motion events. The segmentation window between the two low motion events can then be analyzed for changes in angular rotation of the pelvis in the sagittal plane. If the measured angle rotation is below a certain threshold then no bend event is detected. Once the kinematic signatures meet the requirements to detect a bend, the maximum angular displacement and velocities can be measured. Additionally, the vertical and forward/backward displacement of the pelvis can be calculated. Alternatively, bending events can be detected, segmented, and analyzed when a user is bending downwards to pick up a package. For example, a certain pelvic angular displacement can be used to detect and segment a bend. Bends that have too much pelvic angle changes or vary significantly from a user's normal bending pattern can be shared with the user or manager to avoid injuries before they occur.

Generating a twisting measurement can include detecting the angular rotation at the core trunk of the body. Parameters that help characterize twisting and can be a component of a twisting measurement can include the angular orientation change before and after the twist, the maximum angular velocity measured throughout the twist, duration of twist and the number of twists. Segmentation, detection and twisting analysis can be performed using similar segmentation methods described in the bend analysis above.

In one implementation, generating a twisting measurement can include detecting and segmenting a twisting event, measuring the before and after absolute angle changes of the body, and calculating the maximum angle change velocity throughout the rotation, as well as measuring the time of the entire rotation. Additionally, integrating the angular velocity with error correction can also compute the angular displacement of the rotation. Twisting injury events may occur from excessive twisting and large angular rotation velocities. Twisting events can be detected, segmented, and analyzed to help reduce the maximum angular rotation velocity and to remind users to move with their entire body as opposed to twisting from their hips.

Generating a ladder climbing measurement can include detecting motions used in climbing up or down a ladder. Generating a ladder climbing measurement can include generating parameters related to detecting a climbing up or climbing down event, counting the steps going up or down, quantifying the step time with each step, the double stance time, consistency of each step, and vertical displacement of each step.

In one implementation, generating a ladder climbing measurement can include segmenting each step, detecting a ladder climbing up or down event, counting each step, calculating the vertical displacement up or down, calculating the forward/backward displacement of each step, measuring the time of each step, time consistency of each step, and the consistency of the vertical displacement of each step.

Carelessly climbing down a ladder can result in injury typically near the bottom rungs of a ladder when the worker is not paying much attention and takes a step onto the floor where there is no rung. A generated climbing measurement can be used to remind the user to climb down safely if the speed or the consistency of climbing down increases above a certain threshold.

Actions such as a lifting action or ladder climbing are generally discrete actions and so tracking action ergonomics can include detecting an appropriate action. In one variation, this can include detecting one specific type of action such as a lifting action. In another variation, this can include detecting and classifying multiple types of actions such that lifting, ladder climbing, twisting, bending and other suitable actions can be selectively tracked when they are performed. An action may be detected by detecting an action signature. There could be different action signatures for different types of actions. There could be a lifting action signature, a twisting action signature, a bending action signature, a ladder climbing signature, and the like. The signatures could be detected through analysis of the kinematic data. Alternatively, an action may alternatively be detected when one or more parameters satisfy some condition or threshold. For example, when the knees bend past a certain threshold, the associated data can be considered part of a lifting action. Alternatively, a user or outside system may signal that an action such as a lifting action is being performed. In one variation, sensors in gloves or other equipment can detect when an object is being held or lifted. Various stages of an action could additionally be tracked. Tracking lifting ergonomics can include tracking the approach stage, which is as a user gets in position before lifting, and the lifting stage, which is during the act of lifting. There may additionally be an ending lift stage when a user sets down an item.

Block S224, which includes generating gait biomechanical measurements, functions to characterize aspects of how a worker moves while walking and/or running. Generating gait biomechanical measurements can be based on step-wise windows of the kinematic data—looking at single steps, consecutive steps, or a sequence of steps. In one variation, generating gait biomechanical measurements can include generating a set of stride-based biomechanical signals comprising segmenting kinematic data by steps and for at least a subset of the stride-based biomechanical signals generating a biomechanical measurement based on step biomechanical properties. Segmenting can be performed for walking and/or running. In one variation steps can be segmented and counted according to threshold or zero crossings of vertical velocity. A preferred approach, however, includes counting vertical velocity extrema. Another preferred approach includes counting extrema exceeding a minimum amplitude requirement in the filtered, three-dimensional acceleration magnitude as measured by the sensor. The set of stride-based biomechanical signals can include cadence, ground contact time, braking, pelvic rotation, pelvic tilt, pelvic drop, vertical oscillation of the pelvis, lateral oscillation of the pelvis, forward oscillation, upper body trunk lean, forward velocity properties of the pelvis, step duration, stride or step length, step impact or shock, foot pronation, body loading ratio, foot lift, step and/or stride length, swing time, double-stance time, leg lift response time, activity transition time, stride symmetry, left and right step detection, motion paths, and/or other features. Other health related biomechanical measurements can relate to balance, turn speed, tremor quantification, shuffle detection, variability or consistency of a biomechanical property, and/or other suitable health related biomechanical properties. In one variation, the biomechanical measurements may be generated in a substantially similar manner to those discussed in U.S. patent application Ser. No. 15/282,998.

Cadence can be characterized as the step rate of the participant.

Ground contact time is a measure of how long a foot is in contact with the ground during a step. The ground contact time can be a time duration, a percent or ratio of ground contact compared to the step duration, a comparison of right and left ground contact time or any suitable characterization.

Braking or the intra-step change in forward velocity is the change in the deceleration in the direction of motion that occurs on ground contact. In one variation, Braking is characterized as the difference between the minimum velocity and maximum velocity within a step, or the difference between the minimum velocity and the average velocity within a step. Braking can alternatively be characterized as the difference between the minimal velocity point and the average difference between the maximum and minimum velocity. A step impact signal may be a characterization of the timing and/or properties relating to the dynamics of a foot contacting the ground.

Pelvic dynamics can be represented in several different biomechanical measurements including pelvic rotation, pelvic tilt, and pelvic drop. Pelvic rotation (i.e., yaw) can characterize the rotation in the transverse plane (i.e., rotation about a vertical axis). Pelvic tilt (i.e., pitch) can be characterized as rotation in the sagittal plane (i.e., rotation about a lateral axis). Pelvic drop (i.e., roll) can be characterized as rotation in the coronal plane (i.e., rotation about the forward-backward axis).

Upper body trunk lean is a characterization of the amount a user leans forward, backward, left or right when walking.

Vertical oscillation of the pelvis is characterization of the up and down bounce during a step (e.g., the bounce of a step).

Lateral oscillation of the pelvis is the characterization of the side-to-side displacement during a stride.

Forward velocity properties of the pelvis or the forward oscillation can be one or more signals characterizing the oscillation of distance over a step or stride, velocity, maximum velocity, minimum velocity, average velocity, or any suitable property of forward kinematic properties of the pelvis.

Step duration could be the amount of time to take one step. Stride duration could similarly be used, wherein a stride includes two consecutive steps.

Foot pronation could be a characterization of the angle of a foot during a stride or at some point of a stride. Similarly foot contact angle can be the amount of rotation in the foot on ground contact. Foot impact is the upward deceleration that is experienced occurring during ground contact. The body-loading ratio can be used in classifying heel, midfoot, and forefoot strikers. The foot lift can be the vertical displacement of each foot. The motion path can be a position over time map for at least one point of the runner's body. The position is preferably measured relative to the athlete. The position can be measured in one, two, or three dimensions. As a feature, the motion path can be characterized by different parameters such as consistency, range of motion in various directions, and other suitable properties. In another variation, a motion path can be compared based on its shape.

The foot lift can be the vertical displacement of each foot.

Step length is the forward displacement of each foot. Stride length is the forward displacement of two consecutive steps of the right and left foot.

Swing time is the amount of time each foot is in the air. Ground contact time is the amount of time the foot is in contact with the ground.

Double-stance time is the amount of time both feet are simultaneously on the ground during a walking gait cycle.

Leg lift response time is the amount of time it takes for a user to lift their leg when prompted.

Activity transition time preferably characterizes the time between different activities such as lying down, sitting, standing, walking, and the like. A sit-to-stand transition is the amount of time it takes to transition from a sitting state to a standing state.

Stride symmetry can be a measure of imbalances between different steps. It can account for various factors such as stride length, step duration, pelvic rotation, and/or other factors. In one implementation, it can be characterized as a ratio or side bias where zero may represent balanced symmetry and a negative value or a positive value may represent left and right biases respectively. Symmetry could additionally be measured for different activities such as posture symmetry (degree of leaning to one or another side) when standing.

Left and right step detection can function to detect individual steps. Any of the biomechanical measurements could additionally be characterized for left and right sides.

The motion path can be a position over time map for at least one point. Participants will generally have movement patterns that are unique and generally consistent between activities with similar conditions.

Balance can be a measure of posture or motion stability when walking, running, standing, carrying, or performing any suitable activity. Pelvic coronal drop, pelvic transverse rotation and pelvis lateral oscillation values can help measure balance.

Turn speed can characterize properties relating to turns by a user. In one variation, turn speed can be the amount of time to turn. Additionally or alternatively turn speed can be characterized by peak velocity of turn, and/or average velocity of turn when a user makes a turn in their gait cycle.

Biomechanics variability or consistency can characterize variability or consistency of a biomechanical property such as of the biomechanical measurements discussed herein. Cadence variability may be one exemplary type of biomechanical variability signal, but any suitable biomechanical property could be analyzed from a variability perspective.

The above biomechanical measurements can have particular applicability to walking, running, and standing use-cases. Alternative use cases may use alternative biomechanical measurements relating to acceleration, deceleration, change of direction, jump duration, and other suitable properties of performing some activity.

Block S226, which includes generating a posture measurement, functions to measure ergonomic posture during at least one activity state. In one variation, posture can be measured and distinguished between different activities such as sitting, standing, walking, carrying, and the like. A posture measurement preferably looks at the orientation or an inertial measurement unit relative to some calibrated orientation that is indicative of good posture. In one variation, the posture measurement can use the automatic calibration and posture detection as described in U.S. patent application Ser. No. 15/454,514, filed on Mar. 9, 2017, which is incorporated in its entirety by this reference.

In one particular application, producing biomechanical measurements is customized for monitoring imbalances in a workers gait (e.g., a limp). The set of biomechanical measurements generated for a gait as described above may include signals for the right leg and the left leg. The biomechanical measurements for the right and left can be compared to ascertain the nature of an imbalance. For example, the ground contact time for the right leg can be compared to the left leg. The magnitude of the difference in the ground contact time can correspond to the severity of a limp. In addition, the location of the injury can be detected depending on the gait signature. Furthermore, recovery progress can be objectively observed overtime and shared with the safety management team.

Block S228, which includes detecting kinematic state or events, may function to detect different activity states like distinguishing between a carrying gait and a non-carrying gait and/or detecting a fall or accident. Detecting a carrying gait is preferably based on posture during a detected gait activity. When the posture deviates beyond a particular range, while walking that can be used as a condition for detecting a carrying state. Additionally or alternatively a carrying gait may be based on the task properties. For example, if a worker is currently assigned to deliver a package to a house, and the attributes of the task indicate an object above a weight threshold then a carrying gait may be automatically detected and posture during that period can be analyzed relative to proper posture for a carrying gait. Detection of a carrying gait may be used to classify other biomechanical measurements, such as monitoring the lateral displacement of the pelvis as well as the pelvic rotation and coronal drop angles. These metrics correlate with gait instability and may help predict if a worker is carrying a package that was too heavy and may result in injury overtime.

In another variation detection of kinematic events can include detecting a fall, a stumble or other events. Such events preferably have particular kinematic data patterns that can be detected during continuous monitoring. Detection of an event can result in logging of the event, the time, the location, and/or the associated task.

Block S300, which includes generating an ergonomic model that associates task properties and kinematic activity, functions to analyze the collected observations to relate ergonomics with tasks. Generating the ergonomic model can include processing kinematic activity and task property data, and managing a data model of task associated kinematic activity data (e.g., biomechanical measurements). Generating an ergonomic model can include building the ergonomic model on a local device of the worker such as a phone, wearable device, or the worker activity system. Additionally or alternatively, the ergonomic model in part or whole could be stored and managed on a remote system such as in a remote internet-accessible computing system. The data model can be queried, analyzed, and/or otherwise used in driving various applications.

Processing the kinematic activity and task properties can additionally include identifying ergonomic patterns across multiple tasks. In some cases, the ergonomic patterns are additionally characterized across multiple workers. Ergonomic information can be analyzed based on location, task attributes (e.g., type, metadata related to the task), worker attributes, and/or other aspects in real-time and feedback (auditory, haptic, etc.) can be provided. The processing can include any suitable statistical analysis, machine learning/machine intelligence, deep learning, heuristic-based modeling, or other approaches to organizing the data such that it may be applied to transforming the ergonomics of the work force in block S₃₀₀.

During generation of the ergonomic model, the biomechanical measurements can be processed and transformed into ergonomic measurements. These can be ergonomic assessments of biomechanics analyzed from an ergonomic, health, and/or risk stand point. An ergonomic model can additionally relate to other aspects such as productivity or efficiency. In one variation, this can be a classification process where the biomechanical measurements and/or kinematic data are transformed into ergonomic classifications or characterizations. A classification can be an ergonomic quality label (e.g., good, fair, bad, etc.) or a metric (e.g., an ergonomic score between 0 and 10).

In one variation the ergonomic model can be specific to a particular task. There can be an ergonomic model for each class of kinematic activity measurement discussed above such as an action-specific ergonomic model (e.g., a lifting ergonomic model, a stair climbing model, etc.), gait ergonomic model, posture ergonomic model, and/or kinematic state or events ergonomic models (e.g., fall detection or balance ergonomic models).

In one preferred implementation, a lifting ergonomic model for analyzing lifting can be applied. The ergonomic model can assess the “goodness” or quality of the lift, including assessing various factors relating to the lift such as pelvic angular displacement, maximum angular velocity, vertical displacement, time to complete lift and the consistency of each lift. Each lift event can be compared to all other previous lifts of the user or against an average good lift across the entire population. A risk profile can be generated to highlight if the user is fatiguing or potentially susceptible to injury. The ergonomic model can also compare specific lifting qualities across the entire workforce and provide individual feedback to each worker on particular areas for potential improvement or methods to reduce risk of injury. The ergonomic model can also ensure that the user does not perform too many similar lifts at the same time as to avoid repetitive strain injury. Similarly, the cumulative lifting strain can be monitored as the worker performs multiple lifting actions. Individually, they may not warrant concern but cumulatively as a whole may trigger some alert or action.

A gait ergonomic model functions to analyze the walking (or running gait) of a worker and can be applied to detecting injury or irregular gait patterns. The gait ergonomic model can assess various factors relating to the gait mobility quality such as the average cadence of a user, the variation of cadence and the stride asymmetry of a worker. Gait ergonomic model and/or other ergonomic models can help determine if a worker is healthy, tired or injured. For example, the average cadence metric can signify whether a user is moving quickly or slowly, and the cadence variability and stride asymmetry metrics may signify whether the worker is injured or at risk of injury.

In one example of applying a gait ergonomic model, a worker may start out with an average high cadence value of around no steps per minute during the morning and by the end of his shift, may be walking closer to 90 steps per minute. In addition, if the cadence variation and the stride asymmetry values are low, this may signal general fatigue yet a healthy gait. However, if the stride asymmetry values and cadence variability metrics are high, then the user may also be exhibiting signs of injury or pain. Mobility quality can be substantially similar to the mobility quality described in patent application Ser. No. 15/471,958 filed on Mar. 28, 2017, which is hereby incorporated in its entirety by this reference.

In another example of applying a gait ergonomic model, if a factory worker exhibits erratic walking gait patterns such as significant asymmetry in left and right strides, cadence variability and significant pelvic rotations, the system may detect an injury or a high risk state. Furthermore, if the task was to carry a package, the walking gait ergonomic model may help determine if the package is too heavy for the user to carry, or may determine that fatigue is starting to risk injury and the worker should take a break. Irregular gait patterns could be detected by comparing current biomechanical measurements to historical gait related biomechanical measurements or averaged related biomechanical measurements across a larger group which can characterized in the gait ergonomic model.

A ladder climbing ergonomic model functions to analyze the biomechanics and actions of a worker while climbing up and down a ladder to measure the associated risk of ladder climbing events. Metrics that may be analyzed in this ergonomic model include vertical displacement per step, vertical displacement over a specific period of time, step cadence, step consistency, and step time. For example, as the step cadence or variability increases, the risk profile of the event may increase. If a worker is climbing down the ladder twice as quickly as his historical vertical displacement average, the user may get feedback reminding him/her to be more careful while climbing down. One aspect of the ladder climbing ergonomic model can include tracking ladder step count and/or position. A ladder climbing ergonomic model could additionally be used in combination with a twisting, bending, or posture ergonomic model. For example, the leaning of a worker may be used to trigger warning alerts, and the degree of leaning allowed can be variable with the height or position on a ladder.

A twisting ergonomic model functions to analyze the biomechanics and actions of a worker while working on tasks that include significant amounts of body rotation. The ergonomic model can analyze the associated risk profile of a worker over time. Some metrics may include measuring angular rotation, rotation speed, maximum rotation speed, average rotation speed, and variability in angular rotation and rotation speed. For example, if a worker's angular rotation maximum speed increases throughout the day, the ergonomic model can remind the worker to slow down or take a break before the worker gets an injury. In addition, over time and over numerous users, the ergonomic model can predict the time each individual worker should take a break to avoid injury or fatigue.

A posture ergonomic model functions to analyze the biomechanics and actions of a worker's posture while working. This could be particularly applicable when a worker is working on the factory floor. The model can analyze a worker's posture throughout the day and may provide haptic feedback to remind the worker if their posture is in a bad posture position for an extended period of time. Proper posture is important while working on an assembly line or carrying a heavy package. The ergonomic model may quantify the angle of a worker's neck, upper back or lower back to make sure the spine is in a neutral spine posture. Overtime, the system may predict when a user will have back pain due to poor posture and mobility quality and may intervene to offer additional breaks or recommendations to the worker or team manager.

A carrying ergonomic model functions to analyze the biomechanics and actions of a worker while the worker carries packages and other materials and/or goods in the facility environment. Proper posture, balance, and gait quality are measured to ensure the worker isn't carrying a package that is too heavy that may result in injury, fatigue, or falling. Gait mechanics such as pelvic angular rotation, pelvic angular coronal drop, and pelvic lateral displacement are all indicators of unstable gait. This can result in a back injury, a fall injury, damage of goods, etc. For example, larger pelvic rotations and lateral displacements may predict higher risks to injury and accidents. Lower angular rotations, displacements and consistent cadence variability may predict lower risks. Carrying heavier packages may lead to more variability in cadence and pelvic angle rotations, therefore overtime the system may predict what package weight threshold a user may become capable of carrying without risk of injury.

A fall detection ergonomic model functions to analyze and detect a fall event of a worker. Fall events can occur due to poor biomechanics when carrying a package, carrying a package that is too heavy, or climbing down a ladder too quickly. There are a number of indicators that lead to higher risk of falling that the system and device monitors. Pelvic instability which includes large pelvic coronal drop and pelvic rotation values, stride asymmetries in pelvic drop, pelvic rotation, and ground contact time can be leading indicators to identifying persons at risk of falling while walking. Lateral pelvis sway (rocking back and forth from left to right), shuffling gait, low vertical displacement of feet, and sudden changes in body position, activity state and walking mechanics also add significantly to quantifying the risk of a user who is about to fall.

When a worker is exhibiting some or all of these biomechanical indicators, over a certain amount of time, the system may increase the risk profile or automatically label the individual as high risk. The system can also use time of day or location as additional inputs to the model. For instance, there may be a particular location where the user has nearly tripped that can be recorded, as well as the times the user has nearly fallen or has fallen down.

The system can detect that a fall has occurred. When this happens, the motions before the fall, during and after the fall can be analyzed. The motions beforehand can be analyzed or labeled as high risk to help improve the system's high risk prediction models.

The falling speed, impact, vertical distance and other characteristics can be analyzed. False positives such as a sensor dropping onto the floor can be filtered out. The motions after the fall can also be analyzed to characterize the severity of the fall. For example, the sensor can detect if a user continues to move on the floor, is unconscious and not moving, or is able to get back up and walk around. The location data, heart rate and data from other sensors can also be logged.

Once a fall is detected, and the severity of the fall analyzed (e.g., if the person is unconscious or able to get back up), the device can send an alert directly to emergency contacts, emergency response or a safety manager.

The falling characteristics such as falling speed, impact, severity, and location can be shared directly with safety management.

Additionally, the fall can be used to trigger a smart phone application or peripheral device to automatically contact onsite emergency and safety personnel. Every time a fall is detected, the event is logged and the data before the event is labeled with a fall event. Overtime, falls can be predicted at the system level at specific locations, tasks, individuals, and times of the day.

Additionally or alternatively, the method can have a generalized ergonomic model that can assess factors across different ergonomic model types. In this variation holistic ergonomics or other forms of kinematic activity assessment can be applied and analyzed.

In another variation where location of a task is tracked, generating the ergonomic model can include generating an ergonomic map presenting an analysis of ergonomic measurements plotted by location. More preferably, the ergonomic map is an ergonomic risk map that functions to identify and/or highlight positions associated with task related ergonomic risk. Location and/or task properties can alternatively be used in generating any suitable type of biomechanical map, which biomechanical measurement patterns to locations on a map. The ergonomic map can be presented as a two or three dimensional graphical representation of an environment with a representation of ergonomic performance at various positions and/or regions. In one variation, the ergonomic map or an alternative biomechanical map is a data representation that can be used to drive location-based applications. The generation of an ergonomic risk map can be used in evaluating the safety of an environment, assess productivity, and/or to provide preemptive warning when performing a task in that region. In addition, in a productivity-based biomechanical map, the facility productivity can be mapped over time, location and workers and used to help optimize the productivity of workflow in the facility. This can be used as input into the work distribution system or generating operational efficiency recommendations relating to workers and/or environment.

In a related variation, generating the ergonomic model can include generating an ergonomic score for tasks. Ergonomic and/or health risks can be associated with various task properties and attributes.

By correlating ergonomics with location and or task/properties, the method enables ergonomic warnings to be issued to workers independent of active tracking of kinematic activity of a worker. In one implementation, ergonomic risk alerts could be issued to workers purely based on ergonomic score generated for the task. For example, an ergonomic score for a task lifting an object of a certain size after a worker has worked so many hours may indicate a health risk. The worker may receive an alert to make them mindful of their form and/or work could be redistributed to a worker where the health risk is mitigated.

Block S400, which includes optionally applying the ergonomic model, functions to use the ergonomic history to transform or alter the ergonomics, tasks, or environment. Ergonomics may be altered by addressing the ergonomics of one or more workers, by altering the way that work task is generated or assigned, delivering feedback to a worker or other entity, and/or by altering the use of an environment in real-time.

The ergonomic model can be applied in a variety of ways. Multiple types of ergonomic models can be used. Additionally, different tiers of ergonomic models such as a worker specific ergonomic model and a workforce logic model can be used in generating some outcome.

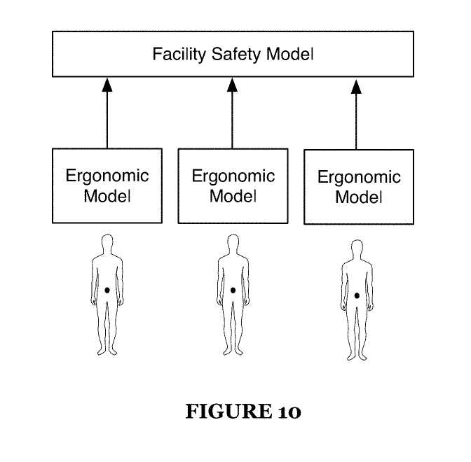

In one implementation, the biomechanical measurements and the task property data can be stored in a data system. Various forms of analysis can be run on the data. Analysis could focus on individual workers, groups of workers, particular tasks, tasks of particular characteristics, workers or tasks in different locations, or any suitable segment of data. Additional data such as supplemental facility-wide event activity data, injury data, operational data, productivity data, and/or other forms of data could be included. Various forms of analysis can be used for updating and/or generating the various ergonomic models. In one variation of an ergonomic model, a higher level workforce logic model can act across a plurality of ergonomic models as shown in FIG. 10. A workforce logic model can be a high level assessment of on one or more ergonomic models of multiple workers and/or other forms of collected data. The workforce logic model can use machine learning, heuristics, or other analysis tools to assess the biomechanics across multiple workers performing different tasks in different locations. The workforce logic model can be associated with ergonomics and used to assess general ergonomics, safety, productivity, and/or other properties of the workforce. Multiple workforce logic models like a facility safety model and a workforce productivity model could be used simultaneously.

In a variation using a workforce logic model, the method can include generating an ergonomic model across multiple workers as a workforce logic model. The workforce logic model can then assess the various ergonomic models to predict safety, monitor productivity, predict optimizations, and make other assessments. In one implementation, the method could include predicting optimal safety solutions across the workforce from a high level ergonomic model like the work force model and individually directing tasks of workers according to predicting an optimized safety model. For example, a worker that is tired could be shifted off a manually intensive job while a rested worker could be assigned to fill that job.

Applying the ergonomic model may include generating an environmental assessment report, automatically distributing work, generating task-based feedback according to predicted ergonomic risk.

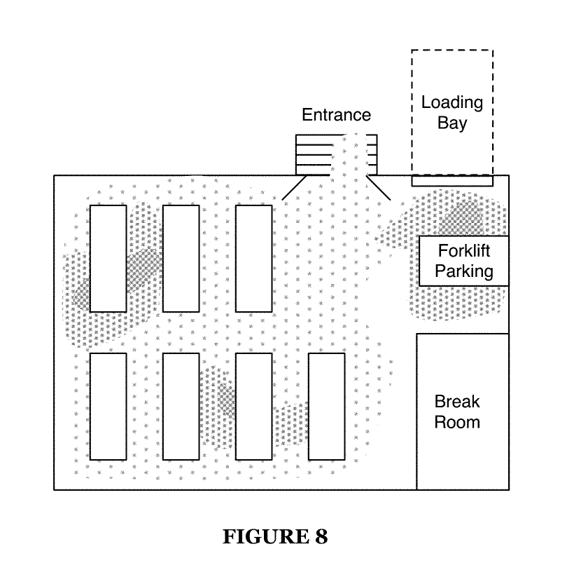

Generating an environmental assessment report, functions to extract patterns in ergonomics concerning the environment, tasks, and workers. In a first variation, generating an environmental assessment report can include generating a graphical map of ergonomic concerns as shown in FIG. 8. The map can be represented as a heat map or any suitable type of visual representation. Generating a map of ergonomic concerns can be static reporting, which may be periodically updated at specified intervals or upon request. Additionally or alternatively, the map can be dynamically updated within a live report that uses substantially live, real-time data. Data from workers can be used in updating the map in substantially real-time where highest priority issues are surfaced at the top.

Automatically distributing work or providing data to floor managers can function to generate and assign work to address ergonomic concerns. Distribution of work can be performed with various objectives such as limiting work, balancing fatigue levels, preventing injuries, minimizing risk, maximizing productivity, and/or other suitable objectives. Distributing work can include a work distribution system assigning a task to at least one of a set of workers based on task properties. Worker state and task specific attributes can be used in determining how work is assigned or distributed across a work force. Work can be assigned based on fatigue within the set of workers. For example, the ergonomics used by a worker may indicate more fatigue relative to other workers. A less strenuous task may be assigned to the fatigue worker and the hard task may be assigned to a better rested worker. In addition, the distribution of work could additionally or alternatively be used for the rotation of tasks within a group of workers. The ergonomic model can be used to predict the time each individual worker should take a break or limit some activity to avoid injury, repetitive stress injuries, and/or fatigue. Additionally or alternatively distributing work can be used based on productivity as indicated and predicted through the ergonomic model. Tasks could be distributed at certain times to certain teams at certain locations to enhance the overall productivity and safety of the facility. Other factors such as experience, job performance, and other worker properties can similarly be accounted for in selecting a worker for a task.

Automatically distributing work can additionally include setting a task specification. Tasks may be augmented based on the way the task is to be performed and the objectives of a task. For example, a task involving transporting an item from one point to a second point in a warehouse may be changed into two tasks: a first worker to move the item to a first mid point and a second worker to move the object from the first midpoint to the final point. In one variation, automatically distributing work can be used to automatically organize a warehouse. For example, during an unloading process, the method can direct workers to move items to storage locations selected based on various criteria, including ergonomic efficiency.

Generating task-based feedback can function to alert a worker to information around ergonomic risk and/or performance of a task. In one variation, generating task-based feedback is according to predicted ergonomic risk, which functions to proactively notify workers of task concerns to preempt bad ergonomics. Furthermore, ergonomic risk models, individual and facility-wide event activity data, injury data, and machine learning models can be used. In one variation, this analysis can be applied to predict future injury events unique to each individual and be leveraged to prevent such injury events from occurring.

Task-based feedback may be audio announcements, haptic feedback (e.g., vibrational alerts), visual indicators, and/or any suitable medium of notifying a worker. With existing approaches of warning messages and stickers, health risk announcements and warnings can become meaningless because their ubiquity causes them to be ignored by workers. In one embodiment, the method can enable dynamic feedback to elevate the level of health risks when necessary based on the current task. Feedback can be triggered based on analysis of various inputs that can be considered independently and/or in combination. Such inputs can include kinematic data, biomechanical measurements, task properties, user location, and/or other inputs.

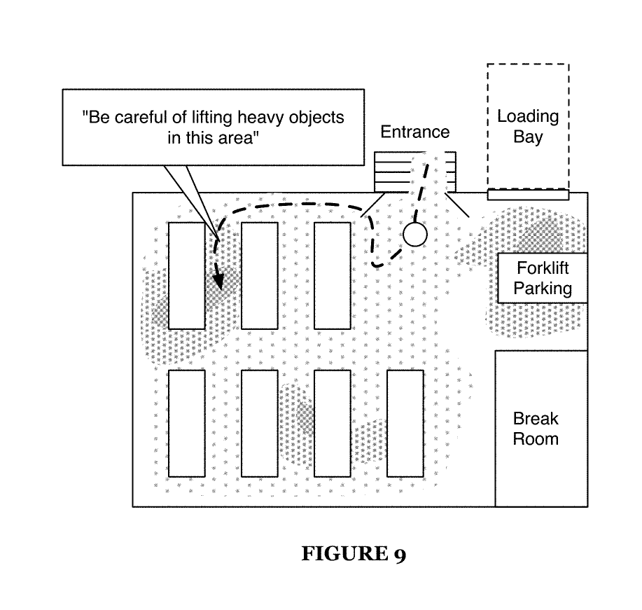

Feedback can be provided preemptively before a worker performs an action. In one variation, generating task-based feedback includes triggering preemptive feedback for the worker on initializing a task assignment based on current location of the worker. The location of a worker, as detected from a positioning system, can be used to detect when the worker is performing a task where other tasks have a higher probability of risk as shown in FIG. 9. In another variation, generating task-based feedback can include triggering preemptive feedback for the worker on initializing a task assignment based on analysis of tasks of similar attributes in the ergonomic model. Task attributes of past similar tasks and their associated ergonomic risk as indicated by the ergonomic model can be used in predicting the ergonomic measurement/risk of the current task and triggering an alert or feedback.

Feedback can be executed to be proportional to a modeled risk assessment. Additionally, the feedback may be provided in a manner that is synchronized with the task. For example, if a worker is tasked with moving a box within a warehouse, and the box is located in an area with a higher than normal ergonomic risk, the worker can receive an audio alert reminding her of proper ergonomics as the worker nears the box location. Task-based feedback can be generated based on group performance and/or individual performance. For example, a worker could be notified when performing a task linked with group ergonomic risk or when working in an area of a sufficiently higher risk profile. In one example, the task-based feedback system may divert a worker from performing a task if the current user is predicted to have high ergonomic risk while performing the task. In stead, the feedback system will identify a different lower-risk task for the worker to perform, and identify another worker who can complete the first task. In another instance, if the system detected the user was climbing down a high risk ladder, the user may receive a haptic or audio alert while climbing down too quickly, as missing the last step often translates to falling and injury. In another scenario, if a specific ladder location has resulted in multiple injuries, workers may always be alerted when climbing down this specific ladder due to the high risk location.

Feedback can additionally be triggered during or after a task. In some variations, a worker could receive real-time feedback on his or her ergonomic analysis after performing an action. This may be used for coaching or training workers so that they can review if they performed a task successfully or unsuccessfully and what can be done to improve their ergonomics during a task. In a coaching variation, generating feedback can include presenting coaching feedback after completion of a task when the ergonomic measurement satisfies a training condition. A coaching condition may be based on the coaching or training history of a worker. For example, a set of different task training objectives can be set, and feedback may be delivered until those training objectives are satisfied. Preemptive feedback and/or coaching feedback can be disabled when the worker completes a training condition.

The application of the ergonomic model is not limited to only workers that have had tracked kinematic activity. The ergonomic model can be used with other workers: workers that have tracked workers or when task properties are tracked for a worker. For example, when distributing work, ergonomic risk alerts may accompany the task assignment, these ergonomic risks may be presented or delivered as preemptive feedback to the worker. The ergonomic risk alerts can be used with workers that have had kinematic activity tracking and with workers that have not undergone kinematic activity tracking. In other words, the kinematic activity tracking of one worker can be used to alter work distribution and/or feedback for a different worker.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A method comprising:
tracking task properties of tasks performed by a worker;
for a set of tasks, tracking kinematic activity of a worker during each task instance, comprising:
at an inertial measurement unit attached to the worker, collecting kinematic data;
processing the kinematic data and thereby generating at least one biomechanical measurement of a task associated kinematic activity; and
generating an ergonomic model that associates task properties and kinematic activity.

2. The method of claim 1, further comprising tracking location of the worker through a positioning system; associating the biomechanical measurement with the location; and wherein generating an ergonomic model comprises generating an ergonomic risk map presenting an ergonomic analysis of biomechanical measurements plotted by location.

3. The method of claim 2, further comprising triggering preemptive feedback for the worker on initializing a task assignment based on current location of the worker relative to the ergonomic risk map.

4. The method of claim 2, wherein tracking location of the worker comprises tracking a location of the worker within a defined environment through a local positioning system.

5. The method of claim 2, wherein tracking location of the worker comprises tracking a location of the worker within a global positioning system.

6. The method of claim 1, wherein tracking task properties comprises retrieving a task assignment from a work assignment system, the task assignment comprising task attributes.

7. The method of claim 6, predicting a biomechanical measurement of a task by analyzing tasks of similar task attributes in the ergonomic model.

8. The method of claim 7, further comprising triggering preemptive feedback for the worker on initializing a task assignment, the triggering of the preemptive feedback being based on analysis of tasks of similar attributes in the ergonomic model.

9. The method of claim 8, further comprising presenting coaching feedback after completion of a task when the biomechanical measurement of a task satisfies a coaching condition; and disabling preemptive feedback and coaching feedback when the worker completes a training condition.

10. The method of claim 1, further comprising delivering a task assignment to a second worker, wherein an ergonomic risk assessment accompanies the task assignment based on the ergonomic model.

11. The method of claim 1, further comprising presenting coaching feedback after completion of a task when the biomechanical measurement satisfies a coaching condition.

12. The method of claim 1, wherein processing the kinematic data and thereby generating at least one biomechanical measurement comprises generating a posture measurement, generating a gait biomechanical signals, and detecting a lifting action and generating a lifting measurement.

13. The method of claim 1, wherein tracking kinematic activity of a worker during a task instance further comprises selecting at least one of a set of kinematic activities, the selection based in part on the task properties; and wherein processing the kinematic data comprises selectively processing the kinematic data according to the selected kinematic activities.

14. The method of claim 13, further comprising tracking location of the worker with a positioning system; and wherein the selection of at least one of a set of kinematic activities comprises selecting a kinematic activity based on a current location of the worker.

15. The method of claim 1, wherein the inertial measurement system comprises a three-axis accelerometer.

16. A method for ergonomic monitoring of a workforce comprising:
tracking task properties of tasks performed by workers of the workforce;
for a set of tasks performed by workers of the workforce, tracking kinematic activity of the set of workers during tracked task instances, where the tracking of kinematic activity for a task instance of a particular worker comprises:
at an inertial measurement unit attached to the worker, collecting kinematic data;
processing the kinematic data and thereby generating at least one biomechanical measurement of a task-associated kinematic activity; and
generating an ergonomic model that associates task properties and kinematic activity across a workforce.

17. The method of claim 16, further comprising, tracking location of the workers;
associating the biomechanical measurements with the location of a worker at the time of the biomechanical measurement; and wherein generating an ergonomic model comprises generating an ergonomic risk map presenting an ergonomic assessment of biomechanical measurements plotted by location.

18. The method of claim 16, further comprising triggering preemptive feedback for a worker on initializing a task assignment based on current location of the worker relative to the ergonomic risk map.

19. The method of claim 16, wherein processing the kinematic data and thereby generating at least one biomechanical measurement comprises generating a posture measurement, generating a gait biomechanical signals, and detecting a lifting action and generating a lifting measurement.

20. The method of claim 16, further comprising predicting safety across the workforce from the ergonomic model; and individually directing tasks of workers according to predicted safety.

21. A system comprising:
worker activity system that includes an inertial measurement system with at least a three-axis accelerometer, the inertial measurement system configured to collect kinematic data, the worker activity system attached to a worker;
processor module that is configured to for a plurality of tasks performed by a worker:
track task properties of tasks performed by a worker,
process the kinematic data and thereby generate at least one biomechanical measurement of a task associated kinematic activity, and
generate an ergonomic module that associates task properties and kinematic activity; and
a user application configured to deliver feedback to the worker.
